(19) United States
(12) Patent Application Publication
(10) Pub. No.: US 2024/0053365 A1
(43) Pub. Date: Feb. 15, 2024

(54) NOVEL BIOMARKER FOR DIAGNOSIS OF ALZHEIMER'S DISEASE, DISCOVERED FROM BLOOD-DERIVED EXOSOMES, AND METHOD FOR DIAGNOSING ALZHEIMER'S DISEASE USING SAME

(71) Applicant: PCL, INC., Seoul (KR)

(72) Inventors: So Youn Kim, Seoul (KR); Gunjoo Park, Seoul (KR)

(21) Appl. No.: 18/254,945

(22) PCT Filed: Nov. 30, 2021
(86) PCT No.: PCT/KR2021/017876

§ 371 Date: Jun. 29, 2023

(30) Foreign Application Priority Data
Nov. 30, 2020 (KR) 10-2020-0164819

(51) Int. Cl. G01N 33/68

(52) U.S. Cl. CPC G01N 33/6896; G01N 2800/2821; G01N 2800/56

(57) ABSTRACT

The present invention relates to a novel exosome-derived biomarker for diagnosis of Alzheimer's disease, a composition for diagnosis of Alzheimer's disease containing the biomarker, a kit for diagnosis of Alzheimer's disease including the composition, and a method for providing information for diagnosis of Alzheimer's disease using the biomarker, composition, or kit. The biomarker for diagnosis of Alzheimer's disease provided by the present invention can be widely utilized for effective diagnosis of Alzheimer's disease and further for treatment of Alzheimer's disease since not only the diagnostic rate of Alzheimer's disease can be increased but also the accuracy, sensitivity, and specificity can be increased through subdivided diagnosis according to the progression stages (healthy, MCI, and AD) of Alzheimer's disease by using the biomarker.

### TECHNICAL FIELD

The present invention relates to a novel biomarker for diagnosis of Alzheimer's disease discovered from blood-derived exosomes and a method for diagnosing Alzheimer's disease using the same. More specifically, the present invention relates to a novel exosome-derived biomarker for diagnosis of Alzheimer's disease, a composition for diagnosis of Alzheimer's disease containing the biomarker, a kit for diagnosis of Alzheimer's disease including the composition, and a method for providing information for diagnosis of Alzheimer's disease using the biomarker, composition, or kit.

### BACKGROUND ART

Alzheimer's disease, a degenerative brain disease accounting for 60% to 70% of dementia, is the most representative disease among the elderly. Alzheimer's disease occurs in 10% of those aged 65 to 74, 19% of those aged 75 to 84, and 47% of those aged 85 or older, and the onset rate thereof is increasing every year, and the disease has emerged as a major social problem.

The existing products are enzyme-linked immunosorbent assay (ELISA) kits for amyloid R peptide and total tau and phosphorylated tau proteins identified by the studies on Alzheimer's disease so far, which only provide information about the proteins, and are not so helpful in diagnosis.

The market share and awareness of leading products of kits for diagnosis of Alzheimer's disease are insignificant, one biomarker affords low accuracy of diagnosis of Alzheimer's disease, and it is necessary to increase the diagnostic rate by forming a biomarker panel for diagnosis of Alzheimer's disease.

Meanwhile, exosome-derived extracts show a clearer relation between diseases and biomarkers than general plasma extracts and can be highly purified, and studies to discover biomarkers from exosome-derived extracts are being actively conducted.

### DISCLOSURE

### Technical Problem

The present inventors have discovered a novel biomarker for diagnosis of Alzheimer's disease from more reliable blood-derived exosomes, and developed a method for providing information necessary for diagnosis of Alzheimer's disease using the biomarker.

### Technical Solution

A main object of the present invention is to provide a novel biomarker for diagnosis of Alzheimer's disease discovered from blood-derived exosomes.

Another object of the present invention is to provide a method for providing information necessary for diagnosis of Alzheimer's disease using the biomarker, a composition, or a kit.

Still another object of the present invention is to provide a composition for diagnosis of Alzheimer's disease containing an agent for measuring an expression level of the biomarker.

Still another object of the present invention is to provide a kit for diagnosis of Alzheimer's disease including the composition.

Still another object of the present invention is to provide a method for providing information to confirm the progression stage of Alzheimer's disease using the biomarker, composition, or kit.

Still another object of the present invention is to provide use of the biomarker for diagnosis of Alzheimer's disease for the preparation of a composition for diagnosis of Alzheimer's disease.

Still another object of the present invention is to provide use of the composition for diagnosis of Alzheimer's disease to diagnose Alzheimer's disease.

### Advantageous Effects

The biomarker for diagnosis of Alzheimer's disease provided by the present invention can be widely utilized for effective diagnosis of Alzheimer's disease and further for treatment of Alzheimer's disease since not only can the diagnostic rate of Alzheimer's disease be increased, but also, the accuracy, sensitivity, and specificity can be increased through subdivided diagnosis according to the progression stages (healthy, MCI, and AD) of Alzheimer's disease by using the biomarker.

### BRIEF DESCRIPTION OF DRAWINGS

FIG. 1 is a schematic diagram illustrating a method for providing information to confirm the progression stage of Alzheimer's disease provided by the present invention;

FIGS. 2A and 2B are charts illustrating the results of first and second analyses of exosome sample proteomics, divided into increasing and decreasing markers;

FIG. 3 illustrates six biomarkers (MYH9, TSP1, TERA, APOM, APOC3, and APOA4) whose increase and decrease patterns largely overlap as a result of first and second proteomics analyses;

FIG. 4A is a pattern set with candidates showing a tendency to increase in the order of mild cognitive impairment and Alzheimer's disease, with the lowest difference in expression in a healthy control group;

FIG. 4B is a pattern set with candidates showing a tendency to specifically increase in mild cognitive impairment;

FIG. 4C is a pattern set with candidates showing a tendency to specifically increase in Alzheimer's disease;

FIG. 4D is a pattern set with candidates showing the highest expression in a healthy control group;

FIG. 5 is a schematic diagram summarizing a process of selecting a transitions of a target peptide;

FIG. 6 is a schematic diagram summarizing a process of optimizing a transition of a target SIS peptide;

FIG. 7 is a graph illustrating the results of performing MRM analysis using a final optimized SIS peptide;

FIG. 8A is a graph illustrating the results of confirming and validating the transition expression difference of AD-specific samples through AuDIT analysis:

FIG. 8B is a graph illustrating the results of confirming and validating the transition expression difference of MCI-specific samples through AuDIT analysis;

FIG. 9 is a graph illustrating the results of analyzing individual samples in a training set;

FIG. 10 illustrates ROC curves of biomarker candidates 1 and 5 acquired through individual sample analysis;

FIG. 11 is a graph illustrating the results of analyzing the expression levels of three biomarkers (ITGB3, APOA4, and CRP) according to the progression stages of Alzheimer's disease;

FIG. 12 is a chart illustrating the results of performing a pair test on eight cloned antibodies by sandwich ELISA;

FIG. 13 is a graph illustrating the results of performing a pair test on eight cloned antibodies by sandwich ELISA;

FIG. 14A is a schematic diagram illustrating a process of measuring antigen-antibody affinity;

FIG. 14B illustrates the results of measuring the affinity of antibody 1 to an antigen;

FIG. 14C illustrates the results of measuring the affinity of antibody 3 to an antigen;

FIG. 14D illustrates the results of measuring the affinity of antibody 6 to an antigen;

FIG. 15 is a schematic diagram illustrating a process of developing an Alzheimer's disease diagnostic kit and testing the performance of the kit;

FIG. 16 is a schematic diagram illustrating a biomarker combination included in an Alzheimer's disease diagnostic kit;

FIG. 17 is a schematic diagram illustrating a process of spotting a biomarker and then performing an assay when a kit templated with a marker Tau associated with Alzheimer's disease as an example is constructed;

FIG. 18 is a schematic diagram illustrating the order of spotting of three biomarkers;

FIG. 19 is a photograph illustrating a process of establishing a condition for the shape of a spot according to a difference in concentration of an antibody to be spotted;

FIG. 20A is a photograph illustrating the results of an experiment to search for APOA4 spotting conditions;

FIG. 20B illustrates the results of performing a test on low-titer, medium-titer, and high-titer samples under conditions considered to be most suitable among the APOA4 spotting compositions;

FIG. 21 is a photograph illustrating the results of performing a ITGB3 screening test;

FIG. 22 is a photograph and a chart illustrating the results of performing a spotting test on a CRP marker;

FIG. 23 is a chart illustrating specific spotting compositions for APOA4, CRP, and ITGB3;

FIG. 24 is a photograph illustrating the results of testing conditions for stabilizing spotted antibodies after spotting of three markers;

FIG. 25 is a photograph illustrating the results of performing a blocking test on spotted antibodies after spotting of three markers;

FIG. 26 is a photograph illustrating the results of performing a drying test on spotted antibodies after spotting of three markers;

FIG. 27 is a photograph illustrating the results of testing conditions of a sample diluent;

FIG. 28 is a photograph illustrating the results of testing conditions of a conjugate diluent;

FIG. 29A is a photograph and a chart illustrating the results of performing Western blot analysis on ITGB3;

FIG. 29B is a photograph and a chart illustrating the results of performing Western blot analysis on APOA4;

FIG. 29C is a photograph and a chart illustrating the results of results of performing Western blot analysis on CRP;

FIG. 30 is a photograph illustrating the results of analyzing an actual sample using a finally constructed kit;

FIG. 31 is a graph illustrating the results of correlation analysis for a ITGB3 marker; and

FIG. 32 is a graph illustrating the results of correlation analysis for an APOA4 marker.

### BEST MODE FOR CARRYING OUT THE INVENTION

In order to achieve the objects described above, an embodiment of the present invention provides a biomarker for diagnosis of Alzheimer's disease including a protein selected from the group consisting of APOA4, ITGB3, and a combination thereof.

Through the SG Cap technology, the source technology of the present applicants, it has been developed a multi-biomarker diagnostic kit that can simultaneously detect an existing marker and a newly discovered marker rather than a single biomarker, and it is expected that the accuracy, sensitivity, and specificity of diagnosis, which have been lacking in existing single biomarker kits, can be increased. The contents of the source technology are described in Korean Patent Nos. 10-1274765, 10-0784437, and 10-1547643.

In order to develop a method for objectively evaluating and diagnosing the onset of Alzheimer's disease, the present inventors have conducted various studies to discover protein markers whose expression levels change depending on the onset of Alzheimer's disease among proteins in serum. As a result, the present inventors have confirmed that the expression levels of APOA4 and ITGB3 change depending on the onset of Alzheimer's disease, and discovered APOA4 and ITGB3 as markers.

The term “diagnosis” of the present invention includes an act of determining the susceptibility of a subject to a particular disease or disorder, an act of determining whether a subject currently has a particular disease or disorder, an act of determining the prognosis of a subject suffering from a particular disease or disorder; or therametrics (for example, an act of monitoring the condition of a subject to provide information on treatment efficacy).

In the present invention, the diagnosis of Alzheimer's disease may be interpreted as meaning an act of objectively determining the onset of Alzheimer's disease in a target patient or the progression stage of Alzheimer's disease when the disease has developed.

The term “APOA4 (apolipoprotein A-IV)” of the present invention refers to a type of plasma protein expressed by the APOA4 gene, and may be called apoA-IV, apoAIV or apoA4. After a protein composed of 396 amino acids is expressed from the gene, APOA4, a glycoprotein composed of 376 amino acids, is expressed through a maturation process. In most mammals, APOA4 is known to be expressed in the intestine and secreted into the circulatory system. The information on the specific amino acid sequence of APOA4 or the nucleotide sequence of the gene encoding the protein is reported in a database such as NCBI. For example, the sequence is reported as GenBank Accession Nos. NP 0.031494, NM 007468, NM 000482, and the like.

The term “ITGB3 (Integrin R3)” of the present invention refers to a peptide constituting integrin, one of the cell surface proteins, and the integrin is known to be involved in cell adhesion and cell surface-mediated signal transduction. The information on the specific amino acid sequence of ITGB3 or the nucleotide sequence of the gene encoding the protein is reported in a database such as NCBI. For example, the sequence is reported as GenBank Accession Nos. NM_000212, NM_016780, NP_000203, NP_058060, and the like.

The biomarker for diagnosis of Alzheimer's disease provided by the present invention may further include CRP.

The term “CRP (C-reactive protein)” of the present invention is a ring-shaped pentamer protein found in human plasma, is synthesized in the liver, and is often used as a test tool to determine whether the human body is in an inflammatory state since the concentration of CRP in the blood increases when the human body is in an inflammatory state. The information on the specific amino acid sequence of CRP or the nucleotide sequence of the gene encoding the protein is reported in a database such as NCBI. For example, the sequence is reported as GenBank Accession Nos. NM_007768, NP_031794, and the like.

Another embodiment of the present invention provides a method for providing information necessary for diagnosis of Alzheimer's disease using the biomarker, a composition, or a kit.

Specifically, the method for providing information necessary to determine the onset of Alzheimer's disease of the present invention includes (a) quantitatively analyzing an expression level of a marker protein selected from the group consisting of APOA4, ITGB3, and a combination thereof in a serum sample of a subject other than a human suspected of having Alzheimer's disease; and (b) associating the quantitatively analyzed level of the marker protein with determination of onset of Alzheimer's disease.

The term “subject” of the present invention may include, without limitation, mammals including mice, livestock, humans, and the like, farmed fish, and the like that have or are likely to develop Alzheimer's disease.

In the present invention, any method known to those skilled in the art can be used in the step of quantitatively analyzing the protein expression level. As specific examples, PCR, ligase chain reaction (LCR), transcription amplification, autonomous sequence replication, and nucleic acid-based sequence amplification (NASBA) methods and the like may be used, but the method is not limited thereto. At this time, the amino acid sequence of the marker protein for diagnosis of Alzheimer's disease according to the present invention or the nucleotide sequence of the gene encoding the protein is known in a database such as NCBI, and those skilled in the art can use an appropriate means required for measurement of the protein expression level.

The quantitatively analyzed levels of the respective proteins vary depending on the condition of the patient, it is thus not easy to use only the quantitatively analyzed level of one of the proteins to determine the onset of Alzheimer's disease, and the quantitatively analyzed levels of the respective proteins may be combined and analyzed to determine the onset of Alzheimer's disease.

As an example of the method of combining and analyzing the respective quantitative analysis results for the proteins, a method in which the onset of Alzheimer's disease is determined by using the quantitative analysis level of each protein measured from a serum sample singly or in combination may be adopted.

As another example of a method of combining and analyzing the respective quantitative analysis results for the proteins, a conventional statistical analysis method may be adopted. At this time, the statistical analysis method that can be used is not particularly limited thereto, but as an example, a linear or nonlinear regression analysis method; a linear or nonlinear classification analysis method; ANOVA; a neural network analysis method; a genetic analysis method; a support vector machine analysis method; a hierarchical analysis or clustering analysis method; a hierarchical algorithm using decision trees or Kernel principal components analysis method; a Markov Blanket analysis method; a recursive feature elimination or entropy-based recursive feature elimination analysis method; a forward floating search or backward floating search analysis method; and the like may be used singly or in combination.

The combination of the quantitative analysis results may be performed using a computer algorithm capable of automatically performing the statistical method.

The method for providing information necessary to determine the onset of Alzheimer's disease provided by the present invention may further include a step of quantitatively analyzing the expression level of CRP protein in step (a).

Still another embodiment of the present invention provides a composition for diagnosis of Alzheimer's disease containing an agent for measuring the expression level of the biomarker.

The term “agent for measuring the expression level” of the present invention refers to an agent capable of specifically binding to and recognizing the protein or mRNA encoding the protein or amplifying the miRNA. Specific examples thereof include, but are not limited to, an antibody that specifically binds to the protein, a primer or a probe that specifically binds to the miRNA, and those skilled in the art will be able to select an appropriate agent for the purpose of the invention.

The agent may be directly or indirectly labeled to measure the expression level of the protein or mRNA. Specifically, ligands, beads, radionuclides, enzymes, substrates, cofactors, inhibitors, fluorescers, chemiluminescent materials, magnetic particles, haptens, dyes, and the like may be used as the label, but the label is not limited thereto. As specific examples, the ligands include biotin, avidin, streptavidin, and the like, the enzymes include luciferase, peroxidase, beta galactosidase, and the like, and the fluorescers include fluorescein, coumarin, rhodamine, phycoerythrin, sulforhodamic acid chloride (Texas red), and the like, but the label is not limited thereto. Most known labels may be used as such detectable labels, and those skilled in the art will be able to select appropriate labels according to the purpose of the invention.

The term “antibody” of the present invention refers to a proteinaceous molecule capable of specifically binding to an antigenic site of a protein or peptide molecule, and such an antibody may be prepared by cloning each gene into an expression vector according to a conventional method to obtain a protein encoded by the marker gene, and forming the obtained protein into the antibody by a conventional method. The form of the antibody is not particularly limited, and any polyclonal antibody, monoclonal antibody, or a part thereof having antigen-binding properties is also included in the antibody of the present invention, and may include not only all immunoglobulin antibodies but also special antibodies such as humanized antibodies. The antibody includes functional fragments of the antibody molecule as well as complete forms having two full-length light chains and two full-length heavy chains. A functional fragment of an antibody molecule refers to a fragment having at least an antigen-binding function, and may include Fab, F(ab′), F(ab′)₂, and Fv.

The term “primer” of the present invention is a nucleotide sequence having a short free 3′ terminal hydroxyl group, and refers to a short sequence capable of forming base pairs with a complementary template and serving as a starting point for copying the template strand. In the present invention, the primer used for miRNA amplification may become a single-stranded oligonucleotide that can act as a starting point for template-directed DNA synthesis under appropriate conditions (for example, four different nucleoside triphosphates and polymerizing agents such as DNA, RNA polymerase or reverse transcriptase) in an appropriate buffer at an appropriate temperature, and the proper length of the primer may vary depending on the purpose of use. The primer sequence does not have to be completely complementary to the polynucleotide of the miRNA of the gene or its complementary polynucleotide, and any primer can be used as long as its sequence is sufficiently complementary for hybridization.

The term “probe” of the present invention refers to a labeled nucleic acid fragment or peptide capable of specifically binding to miRNA. As a specific example, the probe may be constructed in the form of oligonucleotide probes, single stranded DNA probes, double stranded DNA probes, RNA probes, oligonucleotide peptide probes, or polypeptide probes.

Still another embodiment of the present invention provides a kit for diagnosis of Alzheimer's disease including a quantification apparatus for measuring the expression level of the biomarker.

The quantification apparatus included in the diagnostic kit of the present invention may measure the expression level of the marker protein. As a specific example, the kit may be an RT-PCR kit or an ELISA kit, but is not limited thereto as long as the expression level of miRNA or protein can be measured.

In this case, the RT-PCR kit may be a kit including essential elements required to perform RT-PCR. For example, the RT-PCR kit may include test tubes or other suitable containers, reaction buffers (with various pHs and magnesium concentrations), deoxynucleotides (dNTPs), dideoxynucleotides (ddNTPs), enzymes such as Taq-polymerase and reverse transcriptase, DNase, RNAse inhibitors, DEPC-water, sterile water, and the like in addition to each primer specific to the gene. A primer pair specific to a gene used as a quantification control may also be included.

Still another embodiment of the present invention provides a method for providing information to confirm the progression stage of Alzheimer's disease using the biomarker, composition, or kit.

Specifically, the method for providing information to confirm the progression stage of Alzheimer's disease of the present invention includes (a) quantitatively analyzing an expression level of a marker protein selected from the group consisting of APOA4, ITGB3, and a combination thereof in a serum sample of a subject other than a human suspected of having Alzheimer's disease; and (b) associating the quantitatively analyzed level of the marker protein with determination of onset of Alzheimer's disease.

In the method, “subject”, “quantitative analysis of protein expression level”, “combination of quantitative analysis results”, and the like are as described above.

The method for providing information to confirm the progression stage of Alzheimer's disease provided by the present invention may further include a step of quantitatively analyzing the expression level of CRP protein in step (a).

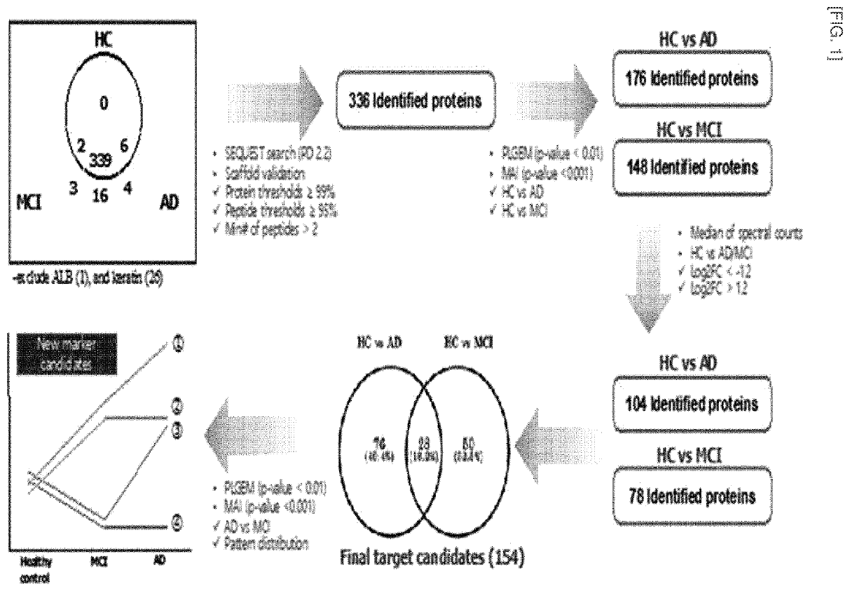

In the method for providing information to confirm the progression stage of Alzheimer's disease provided by the present invention, the progression stages of Alzheimer's disease are healthy control (HC)—mild cognitive impairment (MCI)—Alzheimer's disease (AD), which can be diagnosed in stages as the severity increases. As an example, APOA4 as an MCI-specific marker can be used to diagnose HC, AD and MCI separately, and ITGB3 can be used to diagnose HC and AD separately (FIG. 1).

FIG. 1 is a schematic diagram illustrating the method for providing information to confirm the progression stage of Alzheimer's disease provided by the present invention.

Still another embodiment of the present invention provides use of the biomarker for diagnosis of Alzheimer's disease for the preparation of a composition or kit for diagnosis of Alzheimer's disease.

Still another embodiment of the present invention provides use of the composition or kit for diagnosis of Alzheimer's disease to diagnose Alzheimer's disease.

### MODE FOR CARRYING OUT THE INVENTION

Hereinafter, the present invention will be described in more detail through Examples. However, these Examples are intended to illustrate the present invention, and the scope of the present invention is not limited to these Examples.

### Example 1: Biomarker Screening

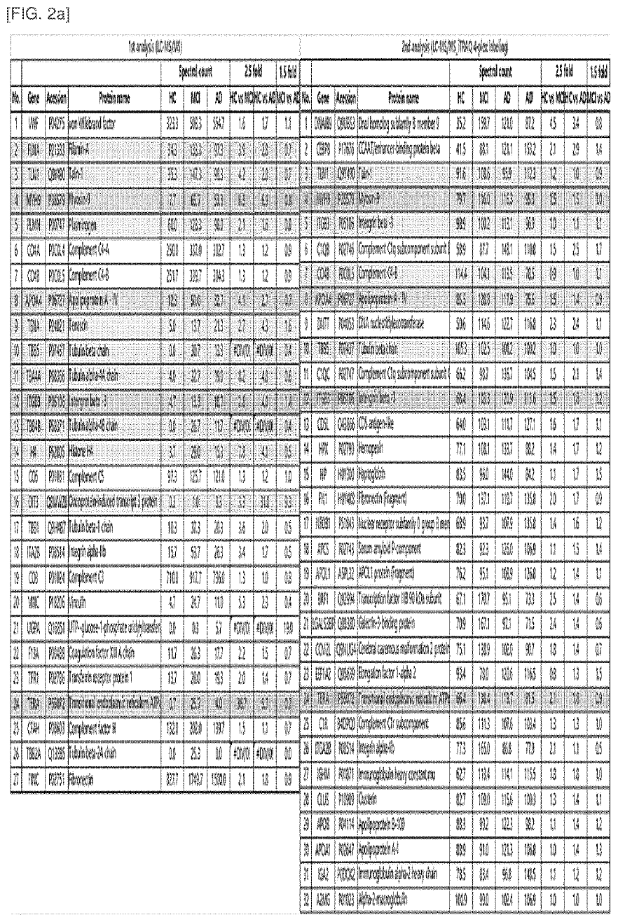

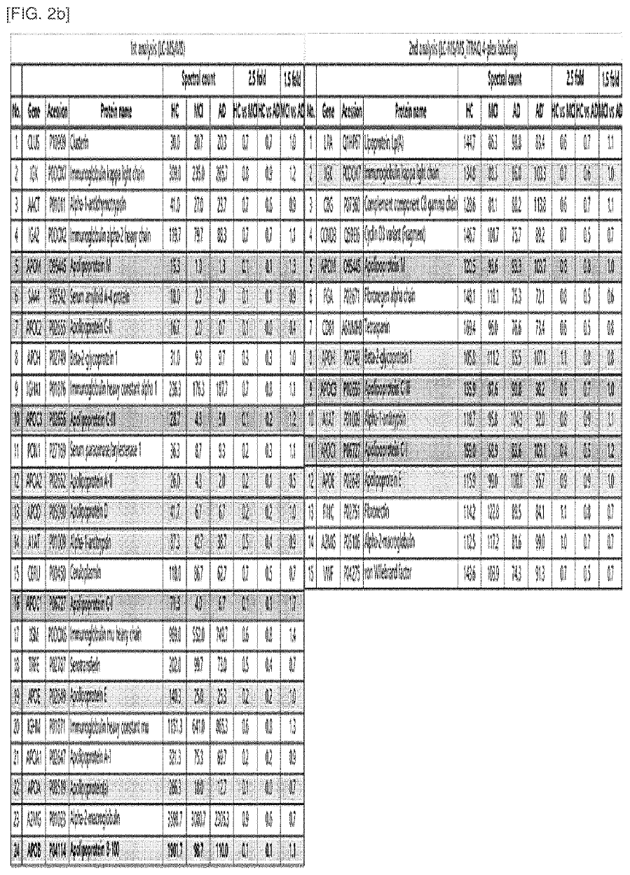

In order to search for candidates for biomarkers predictive of Alzheimer's disease, exosomes were extracted from the blood of a healthy control group, a mild cognitive impairment patient group, and an Alzheimer's disease patient group, and the same samples were subjected to first and second analyses by proteomics. Then, as a result of the first and second analyses, 50 biomarkers with remarkable protein increase and decrease among the healthy control group, mild cognitive impairment patient group, and Alzheimer's disease patient group were selected for each of the first and second analyses. Finally, biomarkers with remarkably overlapping increase and decrease types in the first and second analyses were summarized (FIGS. 2A, 2B, and 3).

FIGS. 2A and 2B are charts illustrating the results of first and second analyses of exosome sample proteomics, divided into increasing and decreasing markers, and FIG. 3 illustrates six biomarkers (MYH9, TSP1, TERA, APOM, APOC3, and APOA4) whose increase and decrease types largely overlap as a result of first and second proteomics analyses.

### Example 2: Validation of Relevance of Biomarkers Selected in Example 1 to Alzheimer's Disease

In order to discover biomarkers for early diagnosis of Alzheimer's disease acquired through Example 1, exosomes were extracted from plasma samples of Alzheimer's disease (AD), mild cognitive impairment (MCI), and healthy persons obtained from Switzerland (Université de Genève. Neurix), and studies were conducted to discover and validate biomarkers for predicting Alzheimer's disease and mild cognitive impairment.

### Example 2-1: Sorting of Expression of Selected Protein Candidates by Pattern Before Biomarker Validation

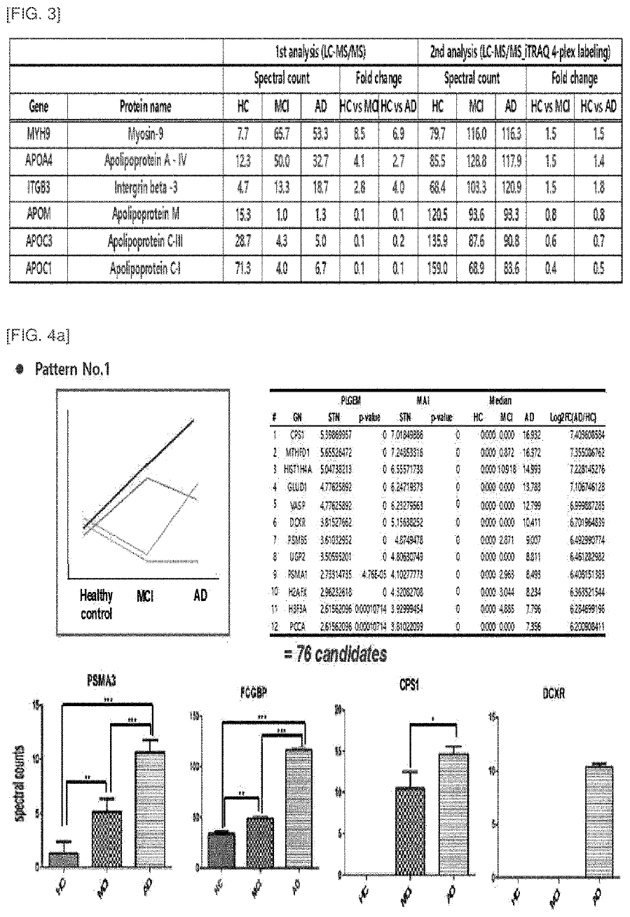

A total of four expression patterns were established by comparing the differences in expression of the selected protein candidates between the Alzheimer's disease and mild cognitive impairment patient groups (FIGS. 4A to 4D).

FIG. 4A is a pattern set with the candidates showing a tendency to increase in the order of mild cognitive impairment and Alzheimer's disease, with the lowest difference in expression in the healthy control group; FIG. 4B is a pattern set with the candidates showing a tendency to specifically increase in the mild cognitive impairment; FIG. 4C is a pattern set with the candidates showing a tendency to specifically increase in Alzheimer's disease; and FIG. 4D is a pattern set with the candidates showing the highest expression in the healthy control group.

### Example 2-2: Selection of Representative Target Peptides and Transitions for Biomarker Candidate MRM Validation

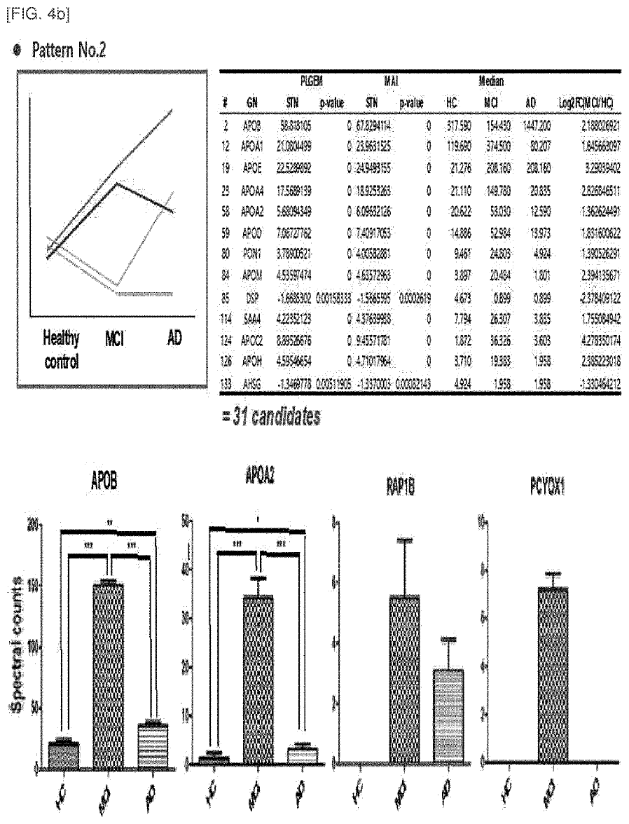

For MRM validation for selected candidates for biomarkers predictive of Alzheimer's disease and mild cognitive impairment, target peptides that could specifically represent each protein were selected. For detection efficiency, peptides containing 6 to 20 amino acids and containing sequences not fragmented by protein post-translational modification or trypsin were excluded. In MRM analysis, the transition, which was a combination of precursor ion (Q1) and product ion (Q3) that expressed quantitative values, was also set in the range of 50 m/z to 1350 m/z according to Agilent, an instrument used in the laboratory, and at least three transitions per one peptide were selected (FIG. 5).

FIG. 5 is a schematic diagram summarizing the process of selecting the transitions of target peptides.

### Example 2-3: SIS Peptide Transition Optimization Process

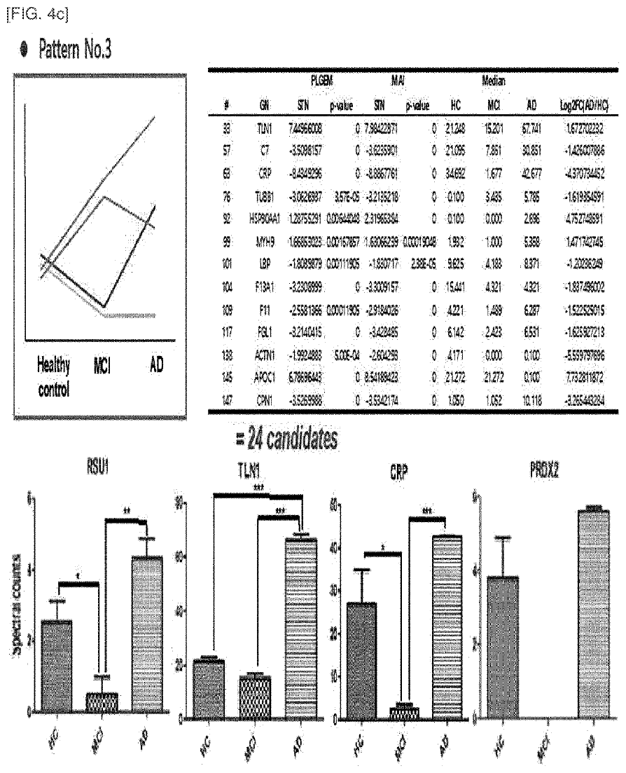

At least three target peptides for each target protein were selected by referring to an online library (SRMAtlas, National Cancer Institute of Cancer Clinical Proteomics, PeptideAtlas). An SIS peptide, which had the same sequence as that of the target peptide but was labeled with isotopes (¹³C and ¹⁵N) on carbon and nitrogen of lysine and arginine, were preferentially synthesized and constructed. Thereafter, the SIS peptide was analyzed, and transition optimization was performed (FIG. 6).

FIG. 6 is a schematic diagram summarizing the process of optimizing the transition of a target SIS peptide.

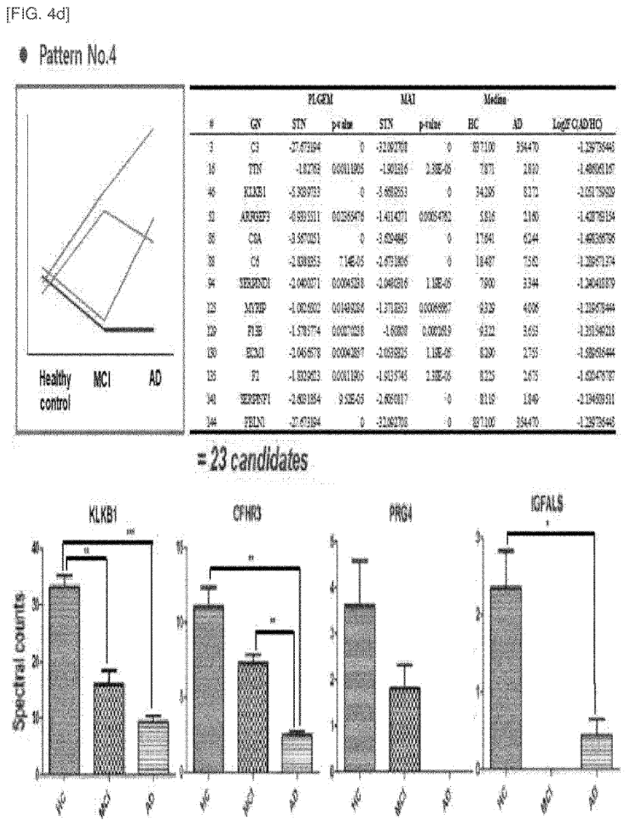

The transition optimized through the SIS peptide was spiked-in to samples pooled for the respective groups of control, mild cognitive impairment, and Alzheimer's disease, and MRM analysis was performed simultaneously. Through simultaneous analysis of SIS peptides and pooled samples, it is possible to determine the exact elution time and standardize absolute quantification. The detected transition was revalidated as a reliable transition during quantification by applying the analytical reproducibility (coefficient of variation, CV<20%) and statistical reference value (p-value<0.05) of the AuDIT (Automated Detection of Inaccurate and Imprecise Transition) program (FIG. 7).

FIG. 7 is a graph illustrating the results of performing MRM analysis using the final optimized SIS peptides.

As a result of AuDIT analysis, 1572 transitions corresponding to 215 peptides were selected as final candidates for individual sample validation.

### Example 2-4: Selection of Final Biomarker to be Used in Kit Development and Validation of Final Biomarker in Patient Sample

In order to increase the reliability of individual sample validation, the analysis was conducted by dividing all samples into two independent cohorts, a training set and a test set. Based on the results of individual sample analysis of the training set, polynomial regression analysis and C-statistic were utilized to analyze the Alzheimer's disease, mild cognitive impairment, and healthy control groups, and the results were used as the basis for biomarker selection, and the final biomarkers were selected in consideration of several other factors specified below.

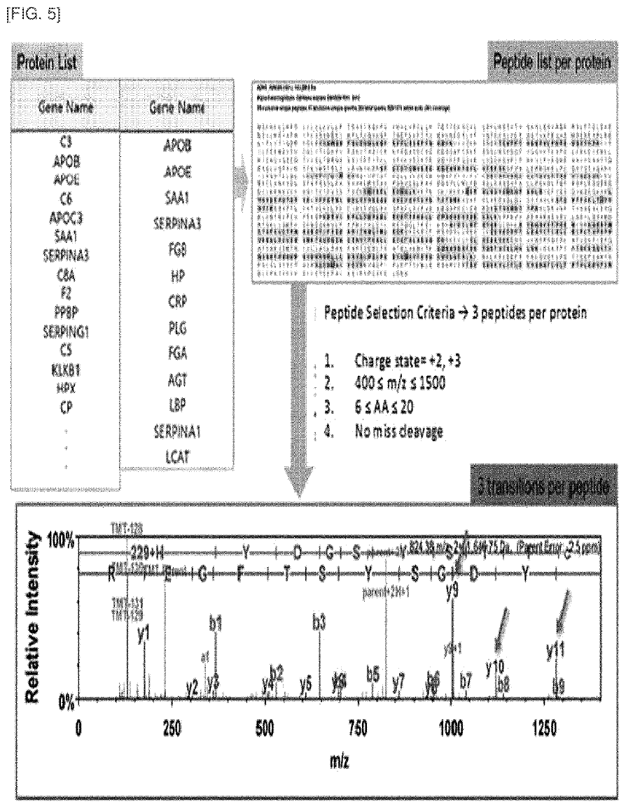

Currently, as a result of analyzing some individual samples, it was confirmed that some candidates showed significant expression differences between the respective disease groups and the healthy control group (FIGS. 8A and 8B).

FIG. 8A is a graph illustrating the results of confirming and validating the transition expression difference of AD-specific samples through AuDIT analysis, and FIG. 8B is a graph illustrating the results of confirming and validating the transition expression difference of MCI-specific samples through AuDIT analysis.

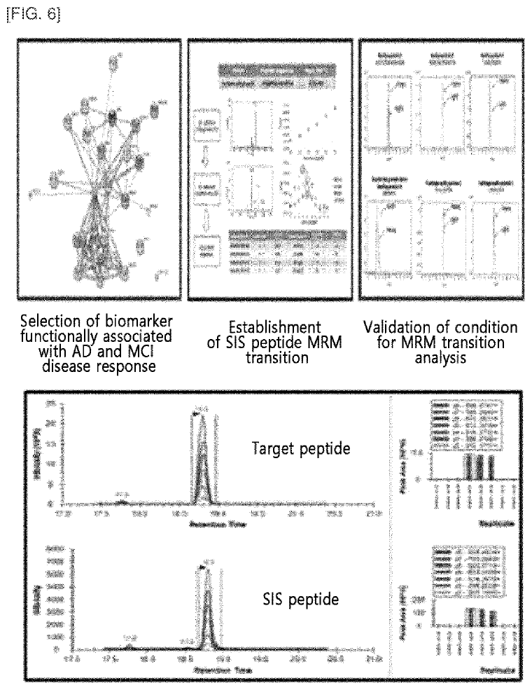

The biomarker candidates were validated by checking the AUC value through the Receiver Operating Characteristic (ROC) curve, and biomarker candidates with an AUC value of 0.7 or more, which was the evaluation standard, were selected. Thereafter, the candidate validated through the training set was revalidated as an Alzheimer's disease-specific biomarker through analysis of individual samples in the test set (FIG. 9).

FIG. 9 is a graph illustrating the results of analyzing individual samples in the training set.

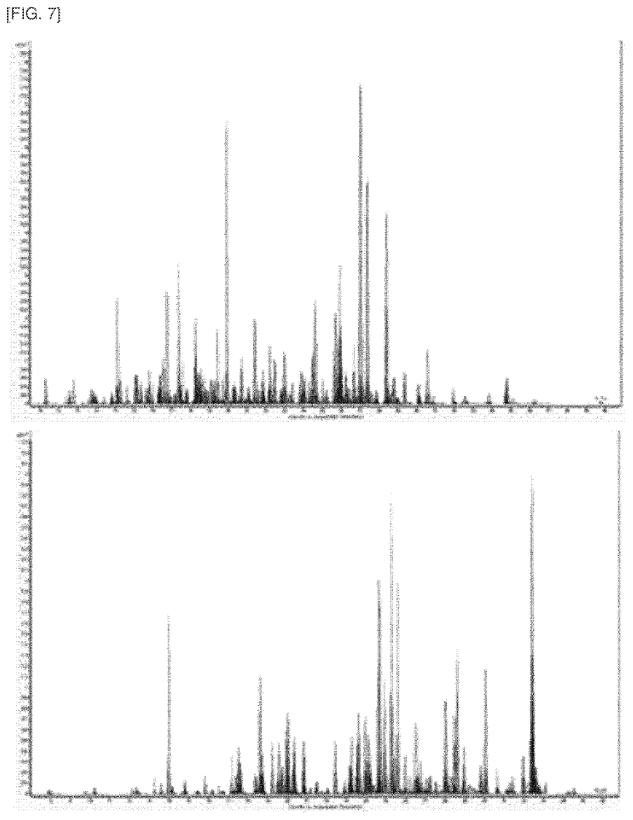

Based on the results of analyzing individual samples, it was decided that MCI-specific biomarker candidate 5 (APOA) would be used as a biomarker in the diagnostic kit. MS/MS results were different even for the APOA biomarkers belonging to the same group, and it was confirmed that particularly APOA4 was MCI-specific (FIG. 10).

FIG. 10 illustrates ROC curves of biomarker candidates 1 and 5 acquired through individual sample analysis.

In the case of AD-specific biomarker candidate 1 (ITGB3), the ROC value was 0.71, and it was decided that the biomarker candidate 1 would be used together with APOA4 in the diagnostic kit. The AD-specific marker was selected as being useful for diagnosis of Alzheimer's disease since its expression level gradually increased as the disease worsened from the healthy control group to the MCI group and AD group.

Finally, the CRP biomarker was also spotted in the same well. CRP failed by a narrow margin to pass the ROC value, which was an internal criterion for selecting biomarkers, but an attempt was made to find significant correlation between Alzheimer's disease and the biomarker when more data were accumulated later, considering that the biomarker had a property of being involved in the inflammatory response and was a marker associated with Alzheimer's disease in the literature.

### Example 2-5: Results of Exosome Proteomics Analysis

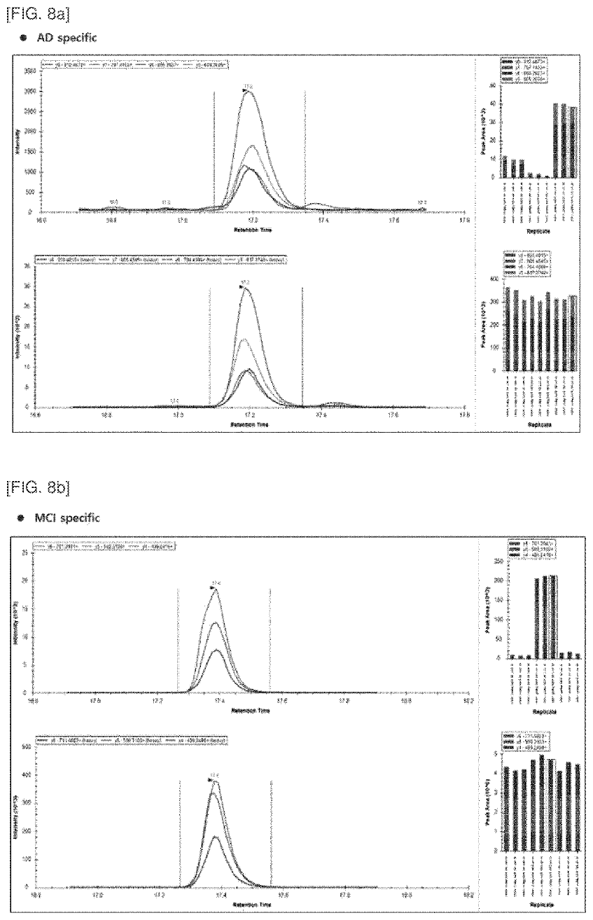

The exosomes (16HC, 9MCI, and 17AD) extracted from the blood of patients at the stages of healthy control, mild cognitive impairment (MCI), and Alzheimer's disease (AD), respectively, when searching for Alzheimer's disease biomarkers were pooled for the same disease stage to prepare three pooling samples for each disease stage, and proteomics analysis of the three biomarkers (ITGB3, APOA4, and CRP) identified above was conducted (FIG. 11).

FIG. 11 is a graph illustrating the results of analyzing the expression levels of three biomarkers (ITGB3, APOA4, and CRP) according to the progression stages of Alzheimer's disease.

As illustrated in FIG. 11, since the expression levels of the three biomarkers showed different patterns for each progression stage of Alzheimer's disease, it was analyzed that the progression stage of Alzheimer's disease could be diagnosed by the expression level patterns of the three biomarkers.

### Example 3: Development of Eight Antibodies to Validated Biomarkers and Selection of Antibody Pairs Through Sandwich ELISA

In order to use APOA4, a selected MCI-specific biomarker, for diagnosis, an antibody was first constructed. Antibodies to ITGB3 and CRP, two biomarkers other than APOA4, were not constructed, but were developed using commercially procured antibodies.

### Example 3-1: Development of Eight Antibodies to Validated Biomarkers

APOA4 antigen was injected into a mouse to induce an immune response, from which a hybridoma was constructed to construct a total of five cloned antibodies, and three cloned antibodies were constructed in the same manner using Goat. For convenience, the eight cloned antibodies were arbitrarily named as antibody 1 to antibody 8, and then reacted with the APOA4 antigen to perform a pair test by way of an ELISA method.

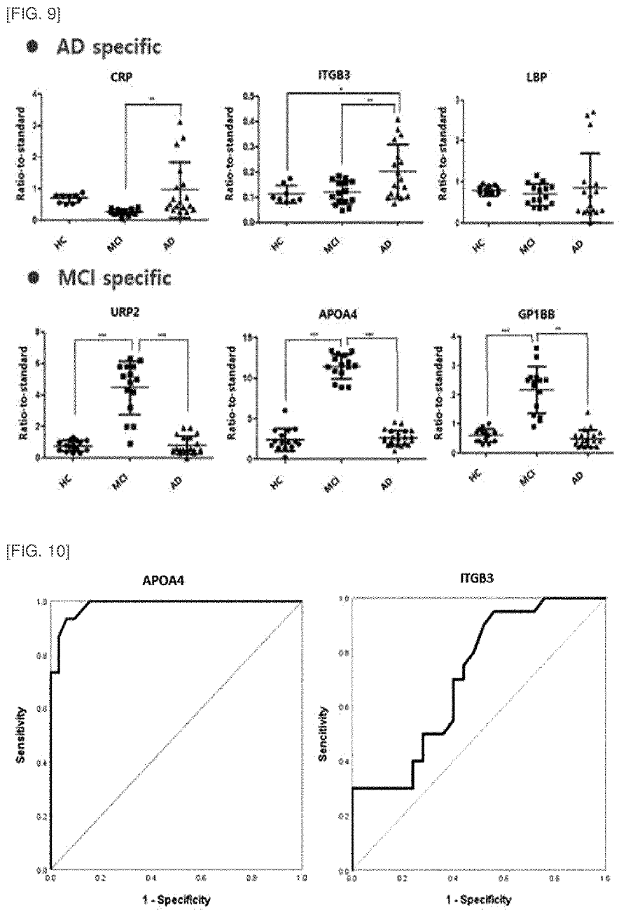

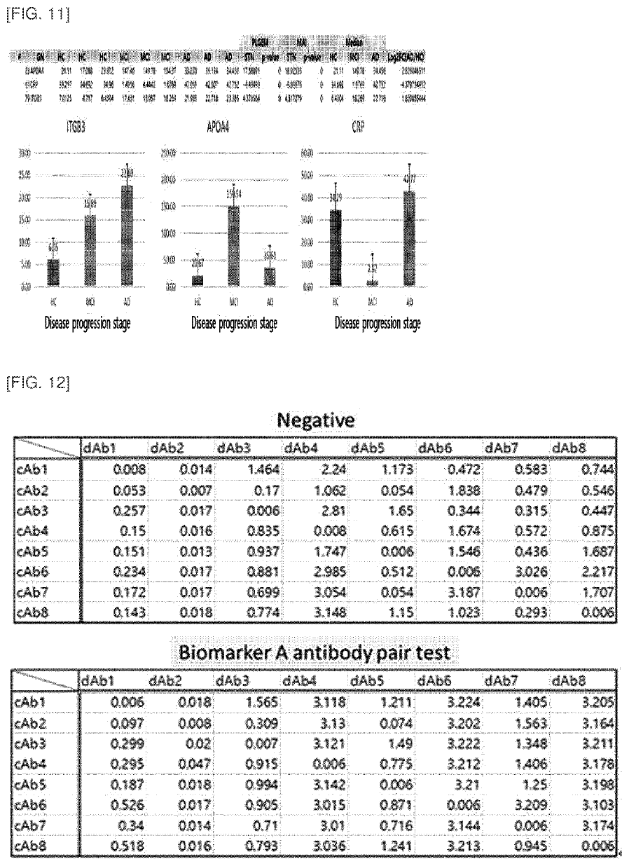

The eight antibodies constructed above were pair tested by sandwich ELISA, and the constructed eight antibodies were tested using Capture and Detection antibodies, respectively (FIGS. 12 and 13).

FIG. 12 is a chart illustrating the results of performing a pair test on the eight cloned antibodies via sandwich ELISA, and FIG. 13 is a graph illustrating the results of performing the pair test.

As illustrated in FIGS. 12 and 13, two pairs considered to be suitable as a pair to be used in the diagnostic kit were selected ((Capture-Antibody 1, Detection-Antibody 6), (Capture-Antibody 3, Detection-Antibody 6)).

### Example 3-2: Affinity Measurement Using Sandwich ELISA and Antibody Pair Selection

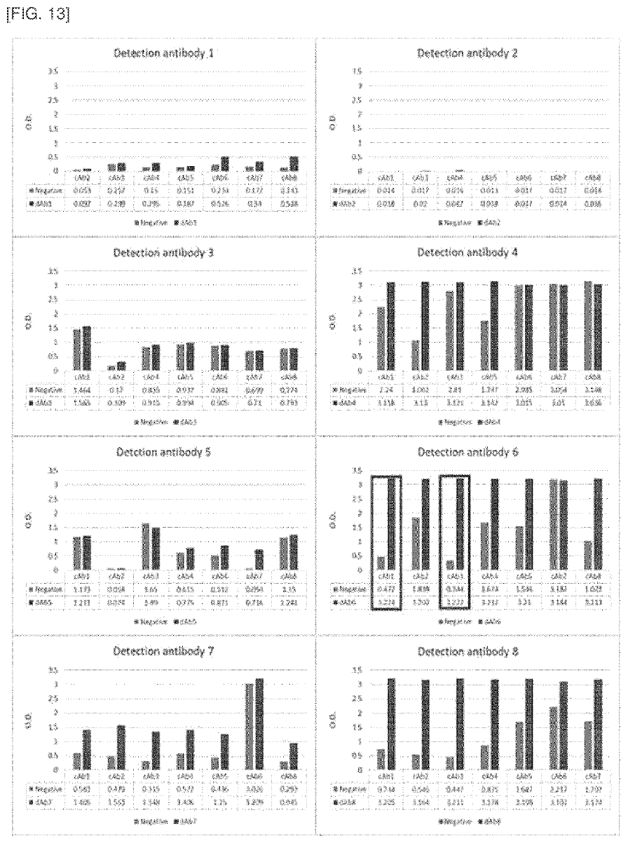

The affinity of antibody 1, antibody 3, and antibody 6 selected as a result of the pair test to the antigen was measured (FIG. 14A). At this time, the antigen used for affinity measurement was the antigen used when the antibodies were constructed. Antigen-antibody affinity was measured for each of the three antibodies (antibody 1-antibody 6, antibody 3-antibody 6). As the basic measurement method, the direct ELISA method was used. Affinity was measured through an experiment inducing competition binding between antigens and antibodies (competition binding assay). The amount of detection antibody to be used during the antigen-antibody competition binding experiment was determined by drawing a Klotz plot graph.

The determined amount of detection antibody and the serially diluted antigen were reacted, the resultant was then dispensed into antigen-coated wells, and fluorescence was measured at 450 nm.

The measured OD value was plotted using Sigma plot software to measure the dissociation constant K_{d} value, which was antigen-antibody affinity.

FIG. 14A is a schematic diagram illustrating the process of measuring antigen-antibody affinity.

### Example 3-2-1: Measurement of Affinity of Antibody 1 to Antigen

The test of affinity of antibody 1 to the antigen was performed by self-assessment (FIG. 14B).

FIG. 14B illustrates the results of measuring the affinity of antibody 1 to the antigen.

As illustrated in FIG. 14B, the dissociation constant K_{d} value acquired using Sigma plot software was 11.38 nM, which was confirmed to be about 10 times less than the affinity of 1 nM or more, the evaluation criterion, and it was found that antibody 1 was not suitable for use in kit development.

### Example 3-2-2: Measurement of Affinity of Antibody 3 to Antigen

The test of affinity of antibody 3 to the antigen was performed by self-assessment (FIG. 14C).

FIG. 14C illustrates the results of measuring the affinity of antibody 3 to the antigen.

As illustrated in FIG. 14C, the dissociation constant K_{d} value acquired using Sigma plot software was 0.2035 nM, which was confirmed to be about 5 times more than the affinity of 1 nM or more, the evaluation criterion, and it was found that antibody 3 was suitable for use in kit development.

### Example 3-2-3: Measurement of Affinity of Antibody 6 to Antigen

The test of affinity of antibody 6 to the antigen was performed by self-assessment (FIG. 14D).

FIG. 14D illustrates the results of measuring the affinity of antibody 6 to the antigen.

As illustrated in FIG. 14D, the dissociation constant K_{d} value acquired using Sigma plot software was 0.2755 nM, which was confirmed to be about 4 times more than the affinity of 1 nM or more, the evaluation criterion, and it was found that antibody 6 was suitable for use in kit development.

Based on the results of the affinity measurement, a diagnostic kit was developed using antibody 3 and antibody 6 as capture and detection antibodies, respectively.

### Example 4: Development of Alzheimer's Disease Diagnostic Kit

### Example 4-1: Outline of Diagnostic Kit

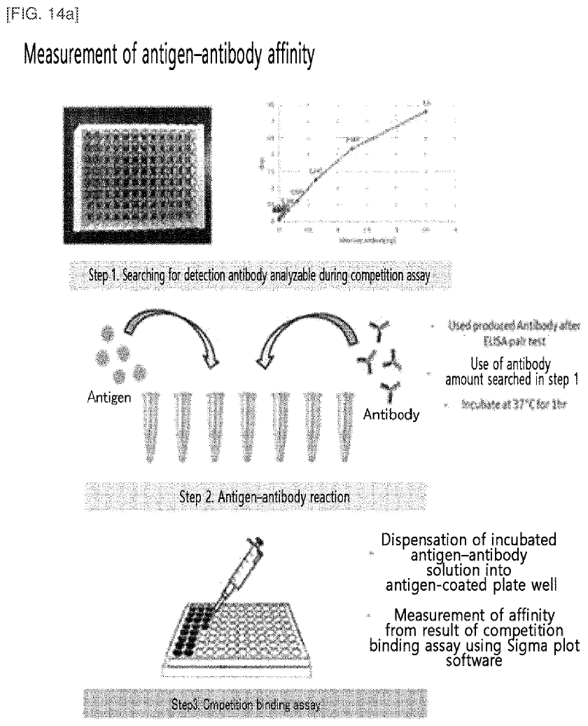

Each step from kit construction to performance test may be classified into three major steps, which are the antibody spotting process, the spotted antibody stabilization process, and the assay reaction process, respectively. Since conditions such as pH and antibody concentration in each step affect the performance of the kit, it may be said that the key to kit development is to search for these conditions and select the optimal conditions (FIG. 15).

FIG. 15 is a schematic diagram illustrating the process of developing an Alzheimer's disease diagnostic kit and testing the performance of the kit.

It was determined that a diagnostic kit using two or more biomarkers was more advantageous in terms of reliability and accuracy of results rather than constructing an Alzheimer's disease diagnostic kit with only one novel biomarker by utilizing the advantage of our company's technology, whereby multiple diagnoses were possible.

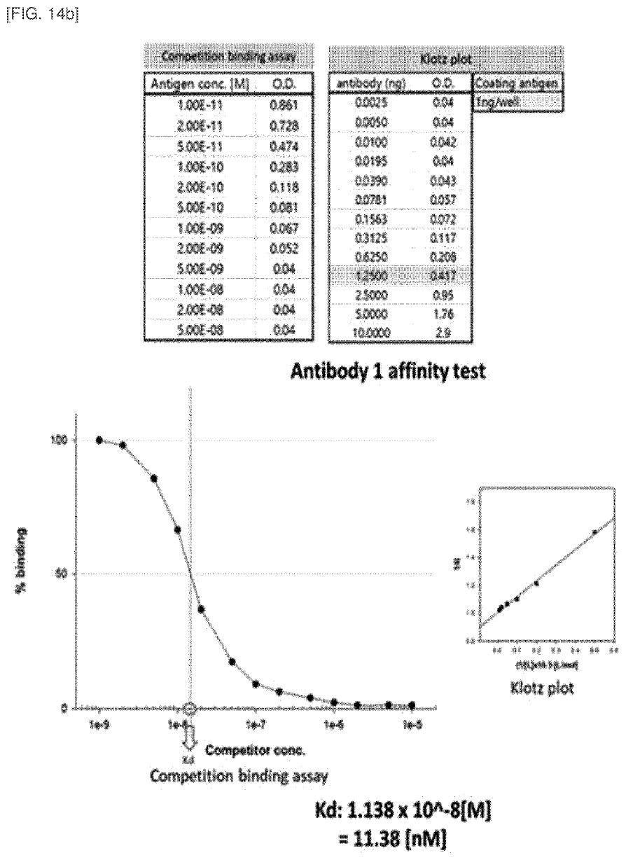

The development goal of the final diagnostic kit was set to multiple diagnoses using a total of three different biomarkers, and the configuration of the three biomarkers was MCI-specific APOA4, AD-specific ITGB3, and CRP, which was expected to be significant in further clinical trials (FIG. 16).

FIG. 16 is a schematic diagram illustrating the biomarker combination included in the Alzheimer's disease diagnostic kit.

### Example 4-2: Spotting of Biomarkers

Spotting work was carried out using antibody 3, which showed superior affinity as a result of the affinity analysis measured in Example 3-2 by the sol-gel method, which was the core technology of PCL Inc.

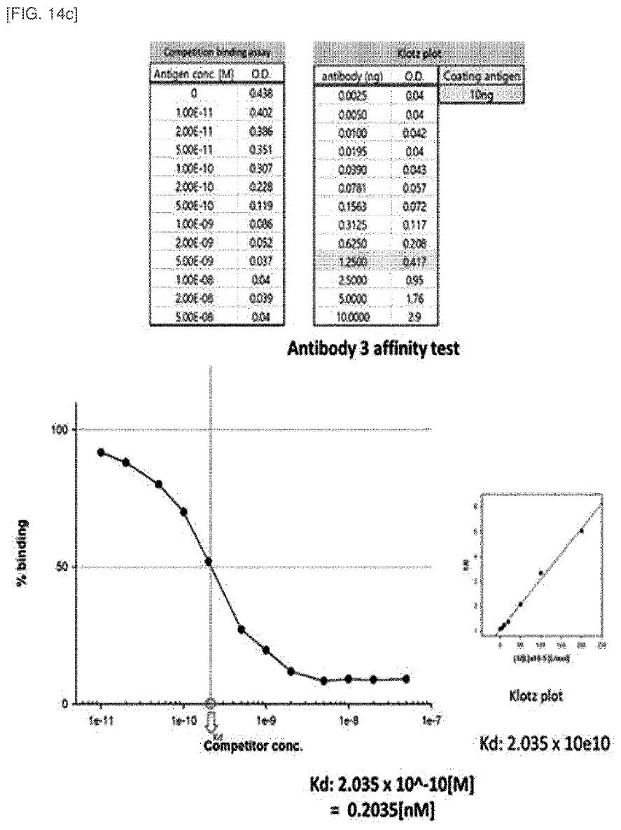

For the spotting process, the capture antibody was spotted on the bottom of the plate well, the antigen and detection antibody were then reacted via a sandwich ELISA method, and the signal value was measured by scanning at 532 nm (FIG. 17).

FIG. 17 is a schematic diagram illustrating the process of spotting a biomarker and then performing an assay when a kit templated with a marker Tau associated with Alzheimer's disease as an example is prepared.

Before use of the actual sample, the antigen used in the assay was assayed with the purchased antigen used at the time of antibody construction, and this was to validate actual patient samples more accurately with a large number of actual samples when the sol-gel spotting process was established later.

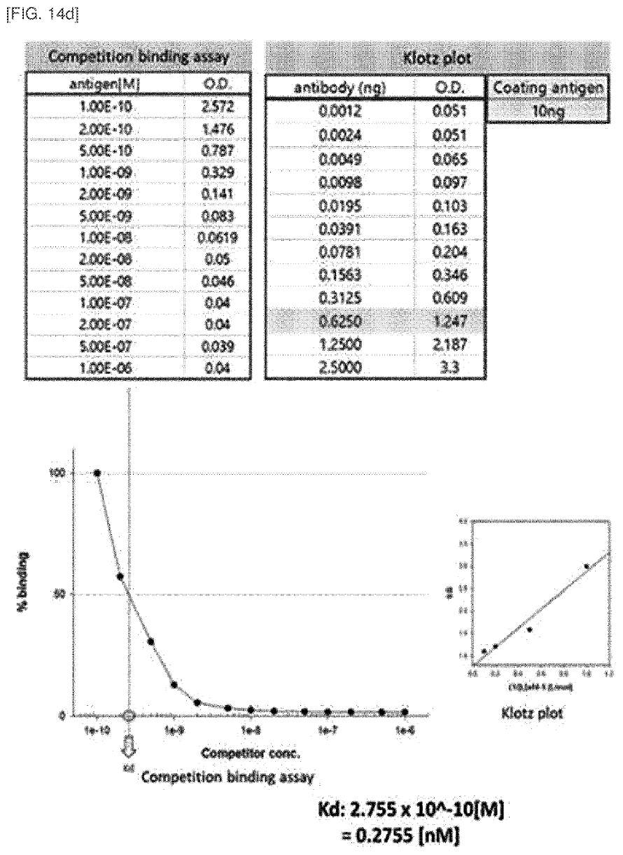

First, the spotting composition was searched for based on the APOA4 marker, which was an MCI-specific biomarker, and when the composition of the APOA4 marker was established, the APOA4 marker was then spotted with ITGB3, and finally all three markers including CRP were spotted together to complete the kit construction (FIG. 18).

FIG. 18 is a schematic diagram illustrating the order of spotting of three biomarkers.

In particular, in the case of APOA4, the antibody on which construction was based was used in the kit construction, and not newly constructed antibodies but commercially procured antibodies were used as spotting and reactive antibodies to ITGB3 and CRP, which were the markers other than APOA4. Antibodies to these two markers were also screened to select suitable antibodies, and the selected antibodies were used.

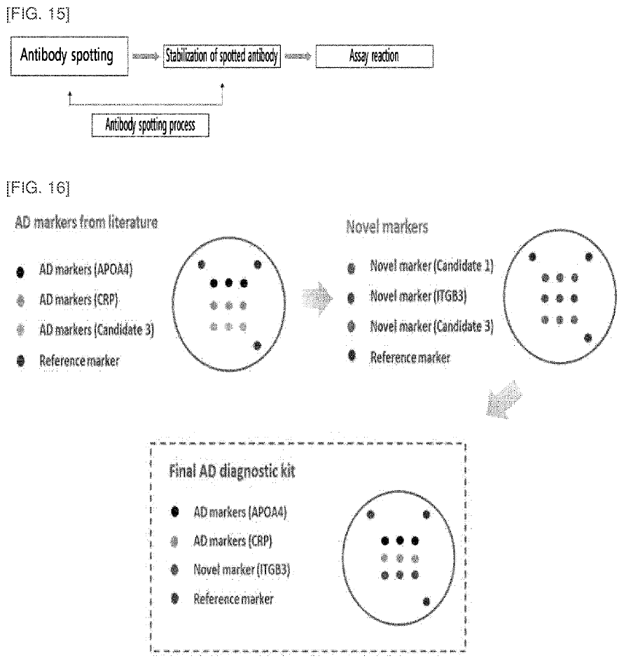

The capture antibody was mixed with sol-gel and spotted according to the protocol established by the present applicants in relation to the composition of sol-gel in advance, and as a result, a phenomenon was observed wherein the spot shrank. It was empirically inferred that the phenomenon was mainly caused by too low a concentration of the capture antibody, and concentration from 0.5 mg/mL, the concentration of the existing capture antibody, to 1 mg/mL was performed, and spotting was performed again at the same sol-gel composition to improve spot shrinkage (FIG. 19).

FIG. 19 is a photograph illustrating the process of establishing the condition for the shape of a spot based on a difference in concentration of the antibody to be spotted.

According to the results of FIG. 19, the concentration of the capture antibody was set to 1 mg/mL to reflect the results in subsequent experiments using various compositions of the antibody and sol-gel spotting.

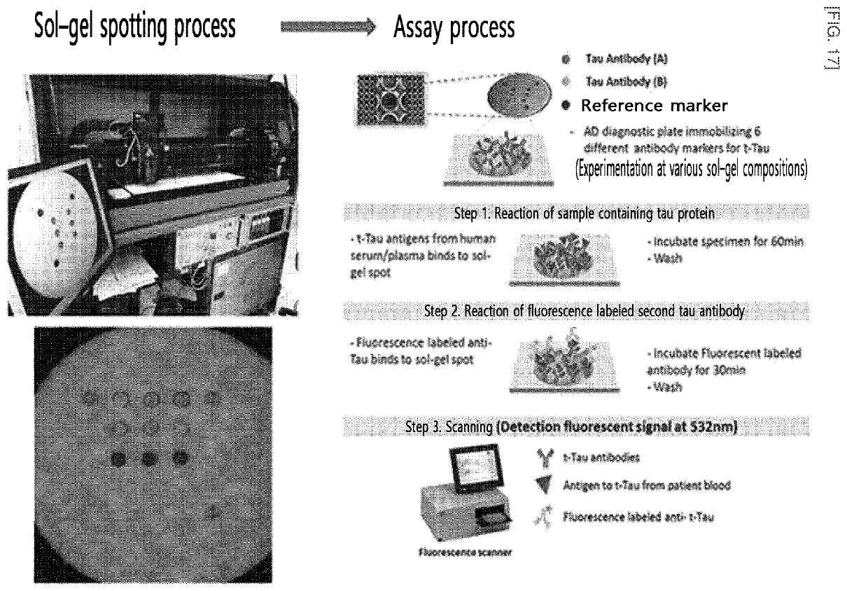

During the spotting experiment of the capture antibody and sol-gel at various compositions of the APOA4 marker, a composition (CBS-3) was found to have a slightly higher signal value (FIGS. 20A and 20B).

FIG. 20A is a photograph illustrating the results of an experiment to search for APOA4 spotting conditions, and FIG. 20B illustrates the results of performing a test on low-titer, medium-titer, and high-titer samples under conditions considered to be most suitable among the APOA4 spotting compositions.

### Example 4-3: ITGB3 Spotting Test

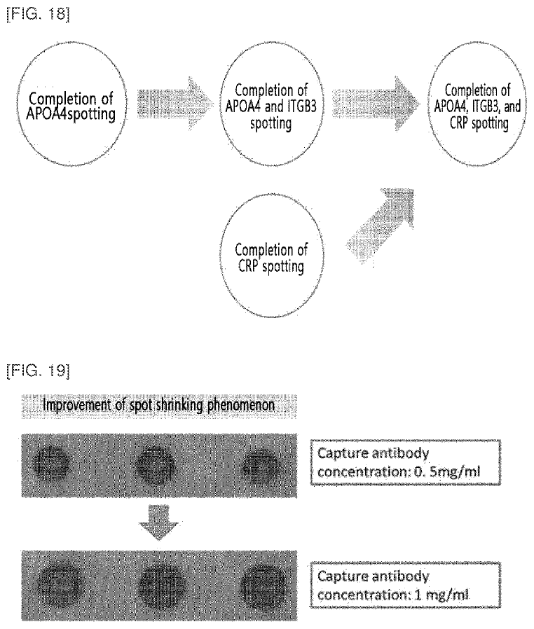

In the case of the ITGB3 marker, a screening test was performed on 10 to 12 spotting compositions according to the protocol established by our company, and through this, a composition considered to be significant was selected. As a result, it was possible to derive a significant signal value from the CBS composition to which nothing was added, and this was selected as the spotting condition (FIG. 21).

FIG. 21 is a photograph illustrating the results of performing the ITGB3 screening test.

### Example 4-4: CRP Spotting Test

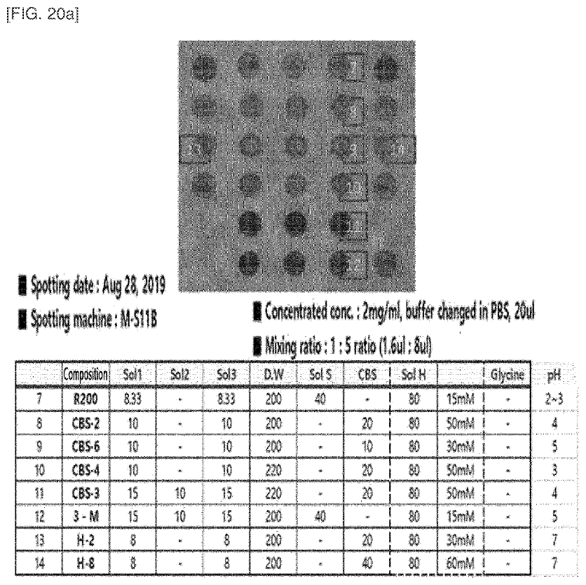

In the case of the CRP marker, spotting was performed using the spotting composition being established as a protocol by our company, as in the case of screening the ITGB3 marker. As a result of the screening test at only the composition, significant signal values were obtained at two compositions, R-1 composition and R-1-1, in which the ratio of sol-gel was quite low. However, in the case of R-1-1, a greatly sticky substance called Sol3 was included among the raw materials for the composition, thus it was considered that there would be difficulties in process or quality control during mass production of the kit in the future, and ultimately the R-1 composition was selected (FIG. 22).

FIG. 22 is a photograph and a chart illustrating the results of performing a spotting test on the CRP marker.

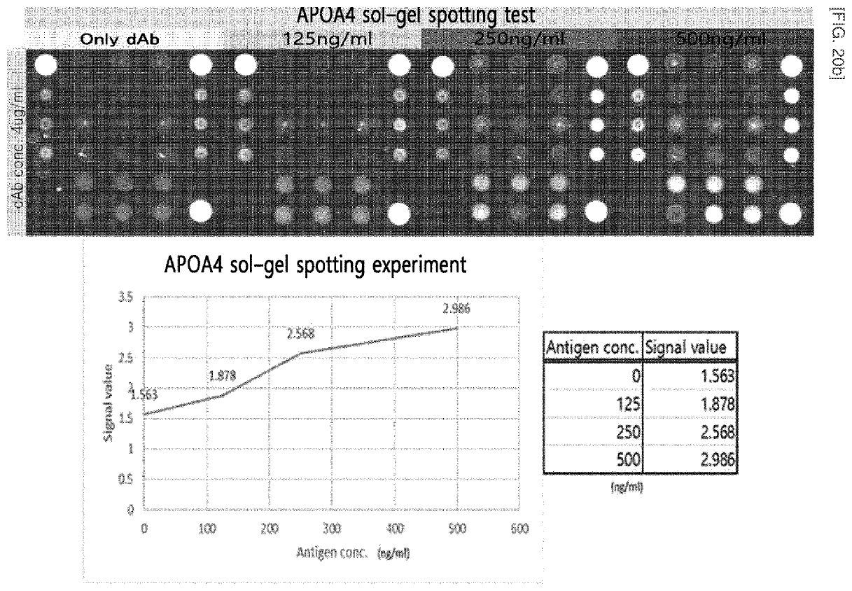

The spotting compositions for APOA4, CRP, and ITGB3 were thus established (FIG. 23).

FIG. 23 is a chart illustrating specific spotting compositions for APOA4, CRP, and ITGB3.

Thereafter, a spotted antibody aging test, a blocking test, and a drying test, which were processes for stabilizing the spotted antibodies, were performed.

### Example 4-4-1: Spotted Antibody Aging Test

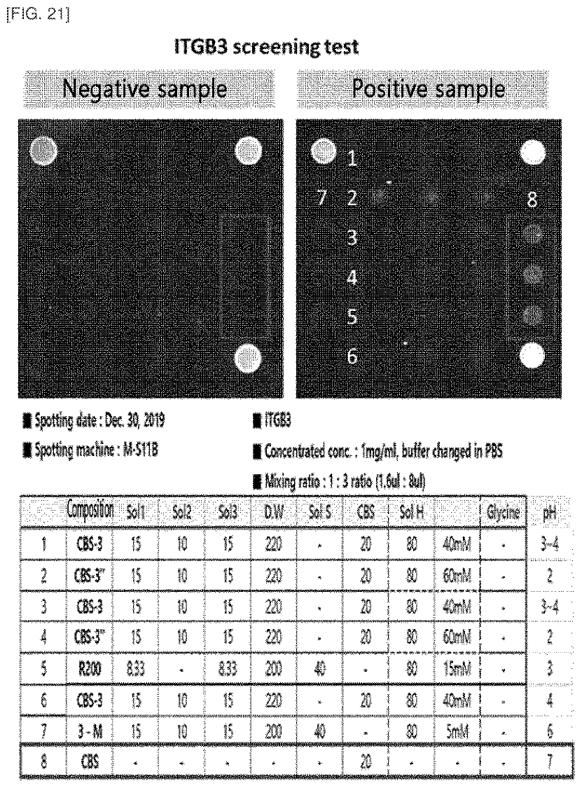

After POA4, ITGB3, and CRP were all spotted, the conditions for the aging step, which was a step to help the spots to be stably spotted, were tested (FIG. 24). Roughly, the aging condition test was performed under two conditions of a production room with a high humidity of about 60% and a desiccator with a low humidity of about 9% and two time conditions of 2 hours and overnight.

FIG. 24 is a photograph illustrating the results of testing conditions for stabilizing spotted antibodies after spotting of three markers.

As illustrated in FIG. 24, it was confirmed that the signal value was high when aging was performed in the desiccator, and the shape of the spot was more stable under the condition of 2 hours than the overnight condition; as a result, the stability of the spot was improved under the condition of a desiccator at room temperature as the aging place and 2 hours as the aging time, and this condition was determined as the aging condition.

### Example 4-4-2: Spotted antibody blocking test

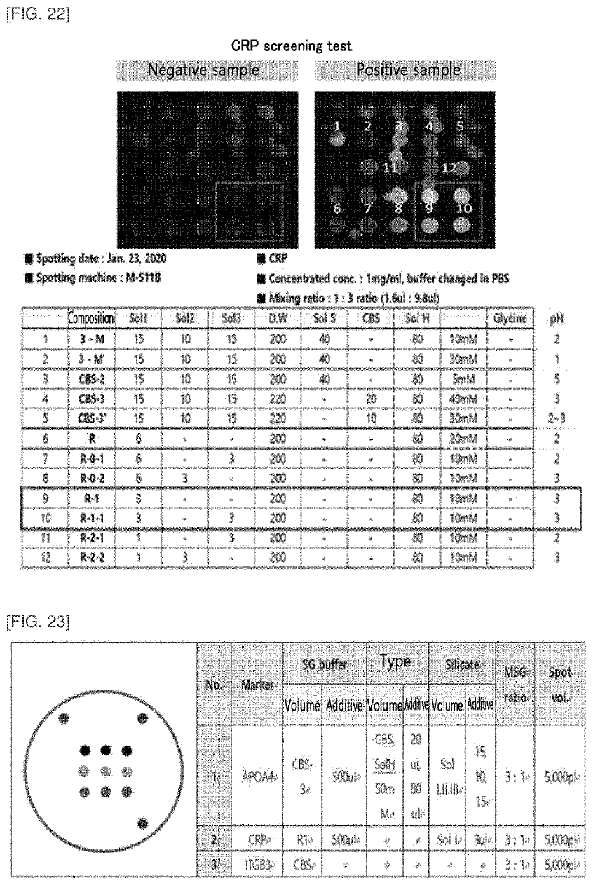

After the aging condition was determined, a blocking buffer test, a process aimed at eliminating non-specific reactions, was performed. The blocking buffer test was performed with a total of three buffers, including the buffer (BSA 1%, Goat serum 0.1%, Tween 20 1 g, Sodium azide 2 g) used in our existing protocol and StabilBlock stabilizers SG01 and ST01 (FIG. 25).

FIG. 25 is a photograph illustrating the results of performing a blocking test on spotted antibodies after spotting of three markers.

As illustrated in FIG. 25, it was confirmed that the most favorable signal was acquired in terms of signal value and spot stability when blocking was performed with ST01.

### Example 4-4-3: Drying Condition Test

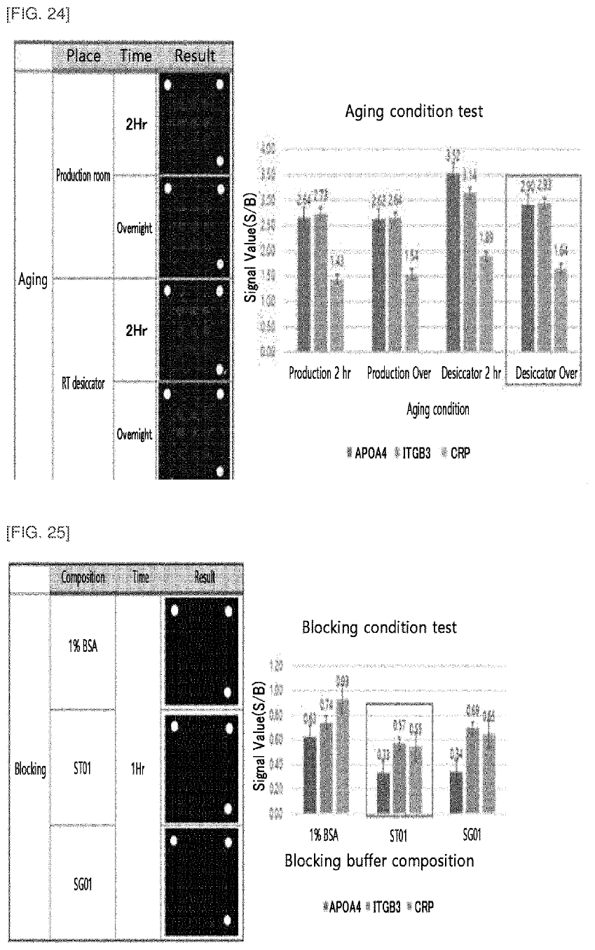

After the blocking process, for the purpose of drying the well, a test was performed under two humidity conditions and two time conditions. The test was performed by setting two conditions of a production room with a humidity of 60% and a desiccator with a humidity of 9% and two time conditions of 2 hours and overnight (FIG. 26).

FIG. 26 is a photograph illustrating the results of performing a drying test on spotted antibodies after spotting of three markers.

As illustrated in FIG. 26, the difference in signal values between drying conditions was not large, but the desiccator-and-overnight condition was determined as the final drying condition in consideration of the fact that the wells were not completely dried under conditions other than the desiccator-and-overnight condition.

### Example 4-5: Diagnostic Kit Final Assay Standardization Test

The criteria for the assay process of the diagnostic kit were set based on the protocol established by our company, and based on this, the optimal assay conditions were selected by modifying the conditions such as the sample diluent and the conjugate diluent.

### Example 4-5-1: Sample Diluent Condition Test

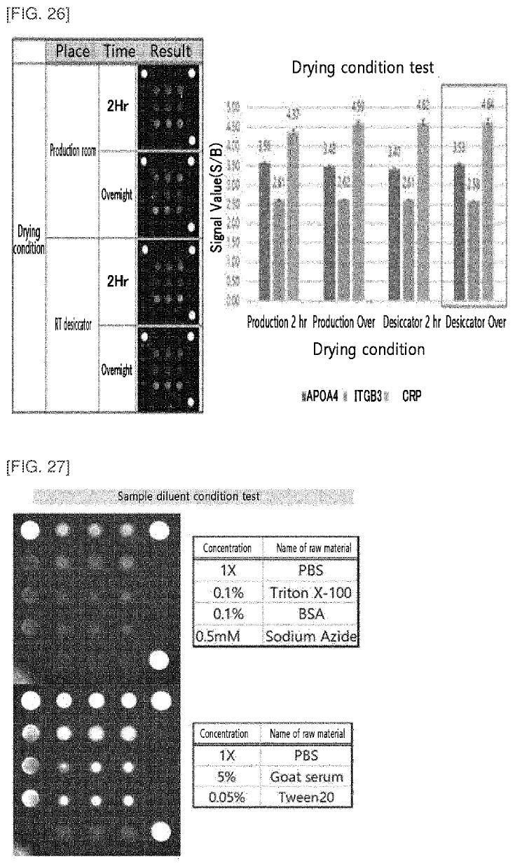

At the time of assay, a condition test was performed for the diluent to be dispensed together with the sample (FIG. 27).

FIG. 27 is a photograph illustrating the results of testing the conditions of sample diluents.

As illustrated in FIG. 27, the reaction was conducted by setting the ratio of the sample to the sample diluent to 1:4 and the total volume dispensed to 100 μL, and as a result, the most improved results were acquired when a sample diluent containing goat serum was used.

### Example 4-5-2: Conjugate Diluent Condition Test

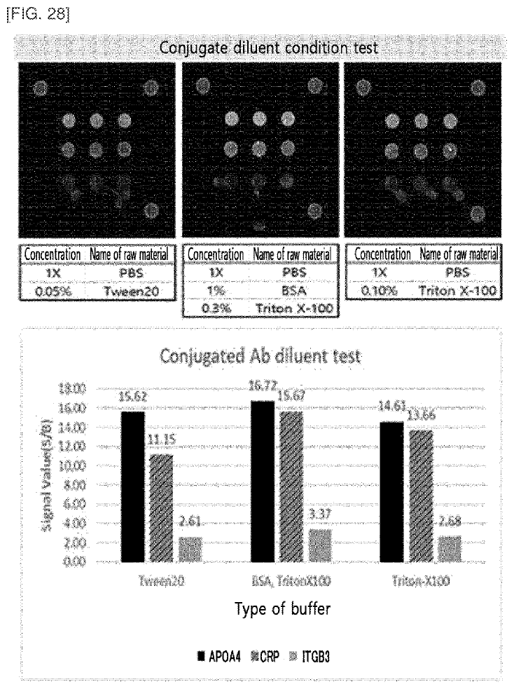

A condition test was performed for the conjugate diluent to be dispensed in the second reaction after the reaction with the sample (FIG. 28).

FIG. 28 is a photograph illustrating the results of testing the conditions of conjugate diluents.

As illustrated in FIG. 28, as a result, it was confirmed that the signal values for all markers increase when the reaction was conducted using a buffer to which BSA was added.

### Example 4-5-3: Diagnostic Kit Final Assay Standardization Information

The final assay standardization information of the diagnostic kit finally confirmed from the results of Examples is as follows:
i. Aging information
1. Room temperature desiccator 2 hr
ii. Blocking information
1. Room temperature 1 hr
iii. Drying information
1. Room temperature desiccator overnight
iv. Driving information
1. Equipment used: Shaker
2. Assay protocol

TABLE 1

| | Sample | | Conjugate | |
|---|---|---|---|---|
| Step | reaction | Washing | reaction | Washing |
| Temperature | 37° C. | | | |
| Volume | 100 μL | | 100 μL | |
| Final amount | Sample: 20 μL | | 5 μg/mL per | |
| | | | marker | |
| Speed | 400 rpm | | 400 rpm | |
| Repetition | 1 | 3 | 1 | 3 |
| Time | 60 min | | 30 min | |
| Buffer | 1X PBS, | 1X Wash | 1X PBS, 1% | 1X Wash |
| | Goat serum | buffer | BSA, 0.3% | buffer |
| | | | Triton X-100 | |

v. Analysis information
1. Filming equipment: Sensovation equipment

### Example 4-6: Final Kit Construction and Actual Sample Test

Three markers were spotted in one well based on the spotting conditions, and a kit performance test was performed using actual samples of HC, MCI, and AD. Among the samples extracted from 380 patients, three to four samples were pooled for each disease stage. Finally, a total of 69 samples were set, 23 for each of the HC, MCI, and AD groups.

### Example 4-6-1: Exosome Western Blot Analysis Results

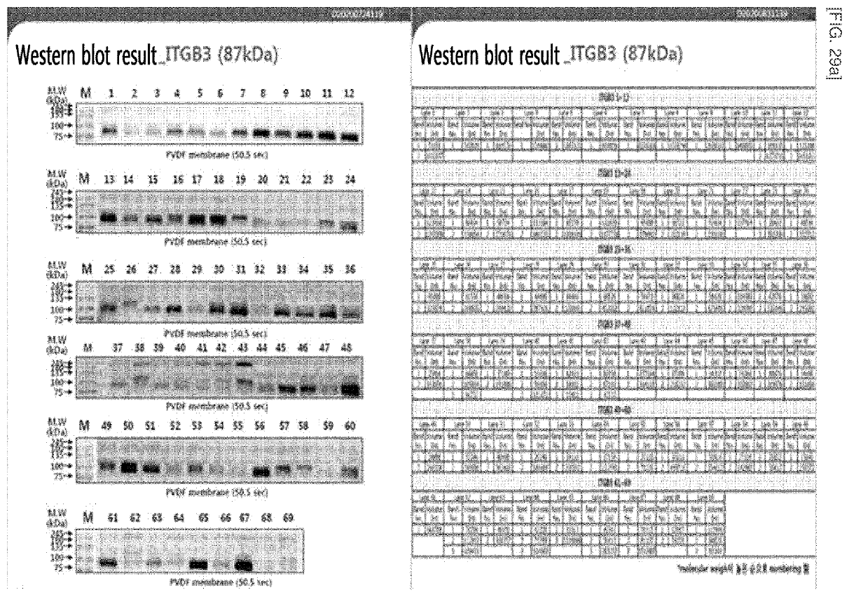

In order to validate the biomarkers APOA4, CRP, and ITGB3, a large number of exosome samples (23 for each stage, total 69) were tested by Western blot analysis. At this time, the order of the samples was arbitrarily set without conducting the experiment by collecting patients for each disease stage in consideration of the difference between gels of Western blot (FIGS. 29A, 29B, and 29C).

FIG. 29A is a photograph and a chart illustrating the results of results of performing Western blot analysis on ITGB3. FIG. 29B is a photograph and a chart illustrating the results of results of performing Western blot analysis on APOA4, and FIG. 29C is a photograph and a chart illustrating the results of results of performing Western blot analysis on CRP.

As illustrated in FIGS. 29A, 29B, and 29C, the WB results corresponded to the proteomics results in the case of ITGB3 and APOA4, but it was difficult to analyze CRP by WB, so the results were attached but were excluded from the correlation analysis in the final conclusion.

### Example 4-6-2: Results of Actual Sample Analysis after Final Kit Construction

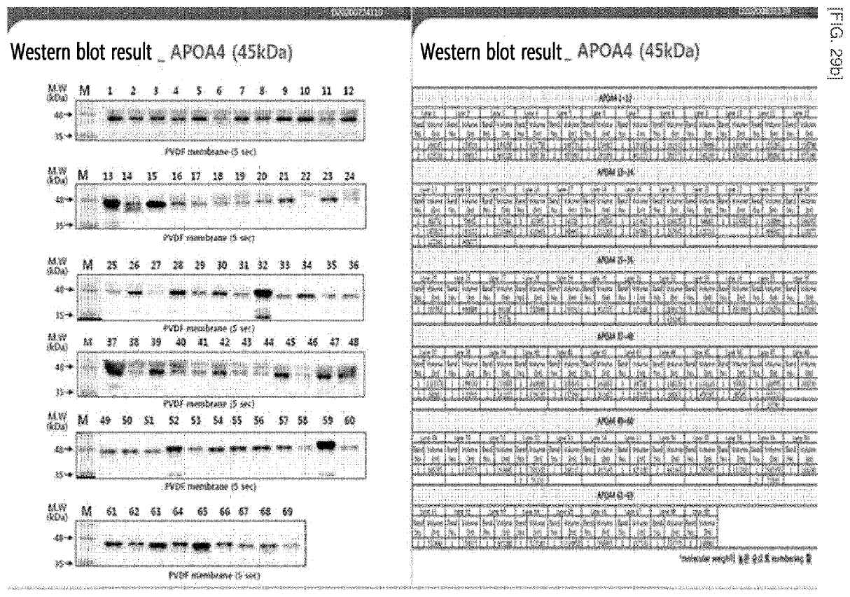

Results were acquired by reacting 69 plasma samples obtained from the same patients as in the Western blot analysis experiment with the constructed kit (FIG. 30).

FIG. 30 is a photograph illustrating the results of analyzing actual samples using the finally constructed kit.

### Example 4-6-3: Correlation Between Actual Sample Analysis Results and WB Analysis Results

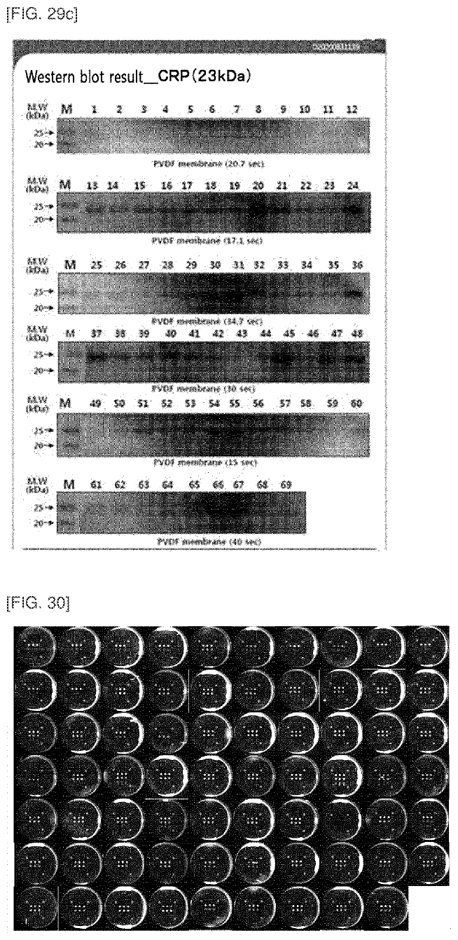

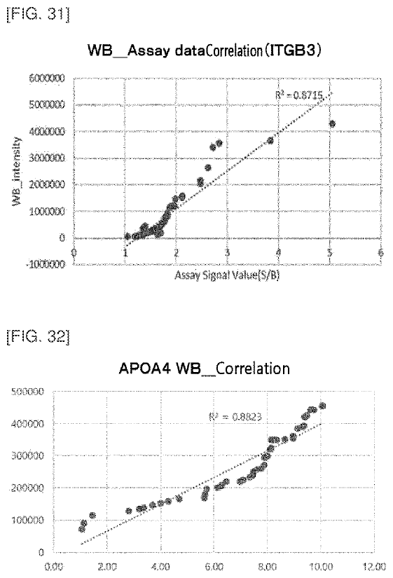

The correlation between the data acquired in Example 4-6-2 and Western blot intensity was compared and analyzed (FIGS. 31 and 32).

First, FIG. 31 is a graph illustrating the results of correlation analysis for the ITGB3 marker.

As illustrated in FIG. 31, in the case of the ITGB3 marker, in all of the proteomics analysis results. Western blot analysis results, and analysis results by the constructed kit, it was confirmed that the protein expression level also tended to increase as the disease progressed from HC to MCI and AD, and the correlation coefficient (R2) was also 80% or more.

Next, FIG. 32 is a graph illustrating the results of correlation analysis for the APOA4 marker.

As illustrated in FIG. 32, it was confirmed that the APOA4 marker had a greatly high expression level in the MCI stage but tended to decrease again in the AD stage.

It is important to promptly diagnose the disease at the MCI stage since the goal of the diagnostic kit is early diagnosis of Alzheimer's disease, and it is possible to clearly know that a patient has Alzheimer's disease without using a diagnostic kit when the patient reaches the AD stage.

It was confirmed that the APOA4 marker showed a correlation coefficient (R2) of about 88% between the analysis results by the constructed diagnostic kit and Western blot in the samples at the MCI stage.

Finally, in the case of the CRP marker, it is difficult to determine the quantitative value in the Western blot analysis, but it can be seen that the expression level of CRP decreases at the MCI stage from the results of proteomics analysis and literature information, and it has been analyzed that it is desirable to utilize the CRP marker as an auxiliary indicator marker.

Based on the above description, it will be understood by those skilled in the art that the present disclosure may be implemented in a different specific form without changing the technical spirit or essential characteristics thereof. Therefore, it should be understood that the above embodiment is not limitative, but illustrative in all aspects. The scope of the disclosure is defined by the appended claims rather than by the description preceding them, and therefore all changes and modifications that fall within metes and bounds of the claims or equivalents of such metes and bounds are therefore intended to be embraced by the claims.

1. A marker composition for diagnosis of Alzheimer's disease, comprising a protein selected from the group consisting of APOA4, ITGB3, and a combination thereof as a marker.

2. A method for providing information necessary to determine onset of Alzheimer's disease, the method comprising:
(a) quantitatively analyzing an expression level of a marker protein selected from the group consisting of APOA4, ITGB3, and a combination thereof in a serum sample of a subject other than a human suspected of having Alzheimer's disease; and
(b) associating the quantitatively analyzed level of the marker protein with determination of onset of Alzheimer's disease.

3. The method according to claim 2, wherein the association step is performed by combining quantitative analysis results of each marker protein.

4. The method according to claim 3, wherein the combination of quantitative analysis results is performed using an analysis method selected from the group consisting of a linear or nonlinear regression analysis method; a linear or nonlinear classification analysis method; ANOVA; a neural network analysis method; a genetic analysis method; a support vector machine analysis method; a hierarchical analysis or clustering analysis method; a hierarchical algorithm using decision trees or Kernel principal components analysis method; a Markov Blanket analysis method; a recursive feature elimination or entropy-based recursive feature elimination analysis method; a forward floating search or backward floating search analysis method; and a combination thereof.

5. The method according to claim 3, wherein the combination of quantitative analysis results is performed using a computer algorithm.

6. A composition for determination of onset of Alzheimer's disease, comprising an agent for measuring an expression level of a marker protein selected from the group consisting of APOA4, ITGB3, and a combination thereof.

7. The composition according to claim 6, wherein the agent is an antibody, primer or probe capable of specifically binding to the marker protein or mRNA of the marker protein.

8. A kit for determination of onset of Alzheimer's disease, comprising a quantification apparatus for measuring expression levels of one or more proteins selected from the group consisting of APOA4, ITGB3, and a combination thereof.

9. A method for providing information to confirm progression stage of Alzheimer's disease, the method comprising:
(a) quantitatively analyzing an expression level of a marker protein selected from the group consisting of APOA4, ITGB3, and a combination thereof in a serum sample of a subject other than a human suspected of having Alzheimer's disease; and
(b) associating the quantitatively analyzed level of the marker protein with determination of onset of Alzheimer's disease.

10. The method according to claim 9, wherein the association step is performed by combining quantitative analysis results of each marker protein.

11. The method according to claim 10, wherein the combination of quantitative analysis results is performed using an analysis method selected from the group consisting of a linear or nonlinear regression analysis method; a linear or nonlinear classification analysis method; ANOVA; a neural network analysis method; a genetic analysis method; a support vector machine analysis method; a hierarchical analysis or clustering analysis method; a hierarchical algorithm using decision trees or Kernel principal components analysis method; a Markov Blanket analysis method; a recursive feature elimination or entropy-based recursive feature elimination analysis method; a forward floating search or backward floating search analysis method; and a combination thereof.

12. The method according to claim 10, wherein the combination of quantitative analysis results is performed using a computer algorithm.
